(19) United States
(12) Patent Application Publication
(10) Pub. No.: US 2026/0000499 A1
(43) Pub. Date: Jan. 1, 2026

(54) Gastroesophageal collar assembly

(71) Applicant: Leameal Junior Davis, II, Ellisville, MO (US)

(72) Inventor: Leameal Junior Davis, II, Ellisville, MO (US)

(21) Appl. No.: 18/759,141

(22) Filed: Jun. 28, 2024

Related U.S. Application Data
(60) Provisional application No. 63/525,662, filed on Jul. 8, 2023.

(51) Int. Cl. A61F 2/04; A61B 17/00

(52) U.S. Cl. CPC A61F 2/04; A61B 2017/00827; A61F 2002/044

(57) ABSTRACT

The gastroesophageal collar being submitted for patent intends to primarily prevent the ascension of the fundus above the diaphragmatic plane and also secondarily to improve the function of the Lower esophageal sphincter, thirdly to reduce occurrences of esophageal reflux and thereby hopefully reducing instances of esophageal cancer. It is estimated as high as %20 of the general population has a hiatal hernia experiencing symptoms of acid reflux and making them at higher risk for esophageal cancer. Currently the standard surgical repair of hiatus is fundoplication. The limitations of fundoplication are its high recurrence rate. It often requires a later second procedure to repair/tighten the fundoplication. By using supple bio-inert materials we intend to create a collar that can be left in place, mechanically preventing the ascension of the fundus and improving LES function. We hope to be the first Collar to prevent hiatal hernia and also improve LES function.

### BACKGROUND OF THE INVENTION

### Field of the Invention

The present invention relates to a gastroesophageal collar assembly for buttressing the lower esophageal sphincter and to a method for preventing the ascension of the fundus above the diaphragmatic plane.

### Brief Description of the Prior Art

Surgical treatment of gastro-esophageal reflux disease consists of performing a fundoplication around the abdominal esophagus to reinforce the lower esophageal spincture. Failure of the surgical treatment is observed in 2-20% of patients (Journal of Visceral Surgery (2018) 155, 127-139) and redo surgery can be difficult.

### BRIEF SUMMARY OF VARIOUS PREFERRED EMBODIMENTS OF THE INVENTION

This section provides a general summary of the disclosure, and is not a comprehensive disclosure of its full scope or all of its features.

In accordance with the various embodiments of the present invention, a prosthetic gastroesophageal collar assembly for buttressing a lower esophageal sphincter includes a collar assembly with a split tubular sleeve having a proximal end and a distal end with an flange. The split tubular sleeve is adapted to grip an outer wall of the esophagus through the diaphragm and the flange is adapted to abut the inner wall of the stomach.

In some embodiments the split tubular sleeve is reinforced with a wire cage and the proximal side of the flange has ruggae. In still other cases. The inside of the tubular sleeve is lined with anti-slip texturing.

In other implementations the prosthetic gastroesophageal collar assembly has an inflatable bladder around the proximal end of the split tubular sleeve.

The prosthetic gastroesophageal collar assembly described above may be used to prevent the ascension of a fundus of the stomach above the diaphragmatic plane after resecting a lower phrenoesophageal ligament by positioning the split tubular sleeve about the base of the esophagus with the flange abutting the lower wall of the diaphragm.

### BRIEF DESCRIPTION OF THE SEVERAL VIEWS OF THE DRAWINGS

In the accompanying drawings in which several of various possible embodiments of the invention are illustrated, corresponding reference characters refer to corresponding parts throughout the several views of the drawings in which:

FIG. 1 is an exploded perspective view of a first prosthetic gastroesophageal collar assembly in accordance with the present invention;

FIG. 2 is a side elevation thereof;

FIG. 3 is a top view thereof;

FIG. 4 is a side view in elevation thereof;

FIG. 5 is a side view in section showing the first embodiment installed at the base of an esophagus;

FIG. 6 is a side elevation of a second embodiment thereof;

FIG. 7 is a side view in section showing the second embodiment installed at the base of an esophagus; and,

FIG. 8 is a side view in section showing a generally normal stomach and esophagus.

### DETAILED DESCRIPTION OF AT LEAST ONE PREFERRED EMBODIMENT OF THE INVENTION

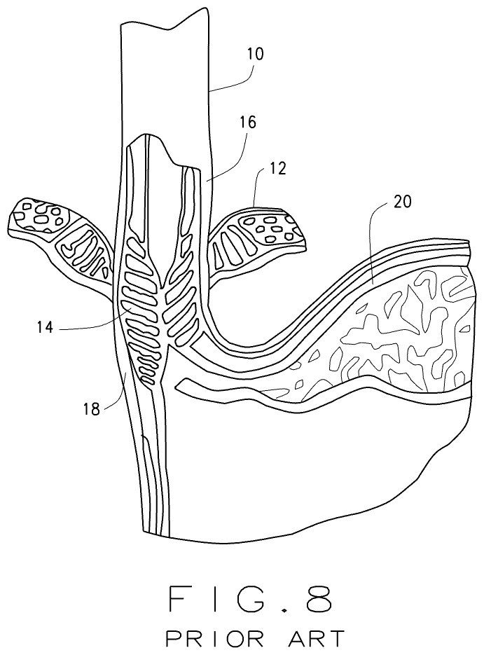

Referring to the drawings more particularly, a normal stomach is shown in FIG. 8 with an esophagus 10 that passes through the diaphragm 12. A lower esophageal sphincter 14 comprised of both smooth and skeletal muscles prevents reflux of stomach contents up the esophagus. The lower esophageal sphincter 14 is supported by upper and lower phrenoesophageal ligaments 16, 18, respectively. When the ligaments weaken, reflux can occur and, in some case, a fundus 20 of the stomach may ascend above the diaphragmatic plane.

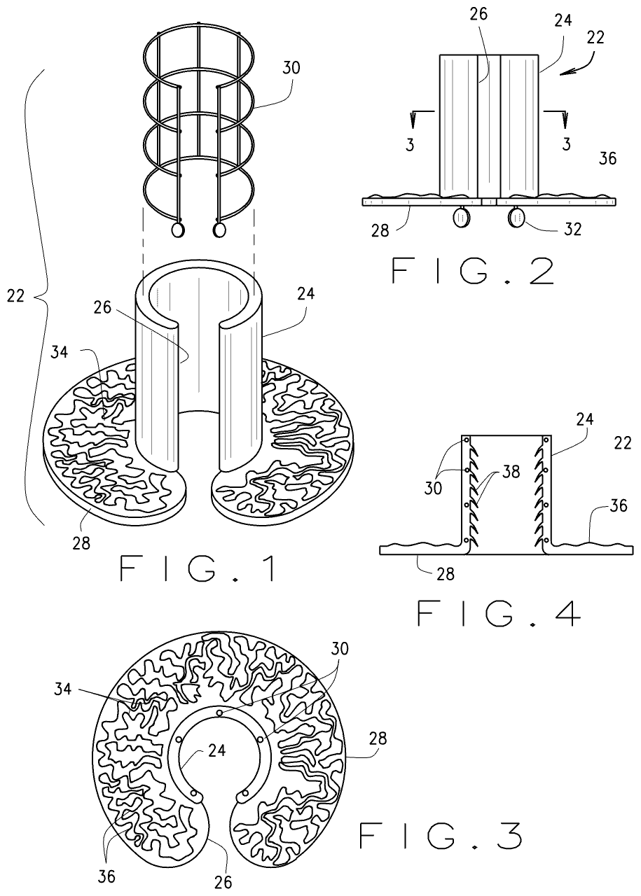

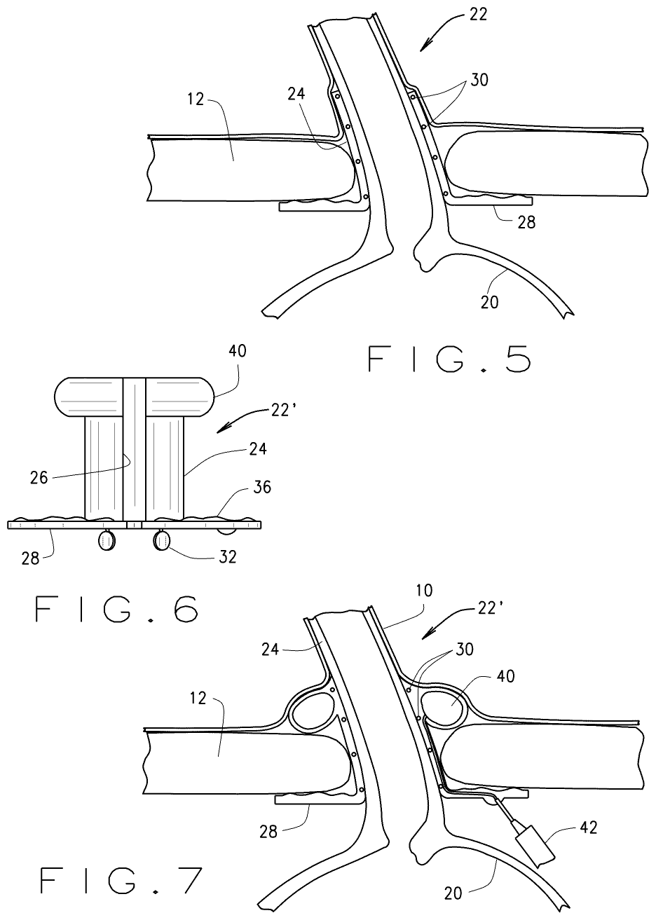

A prosthetic gastroesophageal collar assembly 22 for buttressing the upper esophageal ligaments 16 shown in FIGS. 1-7, includes a split tubular sleeve 24 with a longitudinal slit 26 having a proximal end and a distal end with a flange 28. As used herein, distal refers to a location closer to the stomach and proximal refers to a location closer to the mouth. Tubular sleeve 24 and flange 28 may be formed of a silicone rubber or some other suitable material which will be apparent in view of the present disclosure. In some instances, tubular sleeve 24 may be reinforced with a wire cage 30 with pull tabs or rings 32 at the distal end.

In use, the lower phrenoesophageal ligaments 18 are resected and split tubular sleeve 24 positioned on the lower esophagus 10 with an upper surface 34 of flange 28 abutted against the lower wall of the diaphragm. As shown in FIG. 3, ruggae 36 may be provided on upper surface 34 to prevent possible diaphragmatic spasm.

Upward migration of collar assembly 24 is resisted by flange 28. In one embodiment shown in FIG. 4, tubular sleeve 24 is lined with anti-slip texturing 38 to further reduce the risk of upward migration. In another embodiment as shown in FIG. 7, an inflatable bladder 40 may be provided around the proximal end of tubular sleeve 24. As shown, bladder 40 may be inflated 42 with a saline solution after collar assembly has been installed. Removal of collar assembly 22 if needed may be accomplished by deflating bladder 40 (if present) and pulling on pull tabs or rings 32.

As various changes could be made in the above constructions without departing from the scope of the invention, it is intended that all matter contained in the above description or shown in the accompanying drawings shall be interpreted as illustrative and not in a limiting sense.

What is claimed is:

1. A prosthetic gastroesophageal collar assembly for buttressing a lower esophageal sphincter, said collar assembly comprising split tubular sleeve having a proximal end and a distal end with an flange,
said split tubular sleeve reinforced with a wire cage with tabs or rings at the distal end,
said split tubular sleeve adapted to grip an outer wall of the esophagus through the diaphragm, and
said flange attached to the distal end of tubular sleeve with a proximal side adapted to abut the inner wall of the stomach.

2. The prosthetic gastroesophageal collar assembly of claim 1 wherein the proximal side of the flange has ruggae.

3. The prosthetic gastroesophageal collar assembly of claim 1 wherein the split tubular sleeve has an inflatable bladder around the proximal end.

4. The gastroesophageal collar assembly of claim 1 wherein an inside of the tubular sleeve is lined with anti-slip texturing.

5. A method for preventing ascension of a fundus of the stomach above the diaphragmatic plane, comprising the steps of:
providing a prosthetic gastroesophageal collar assembly for buttressing a lower esophageal sphincter, said collar assembly comprising a split tubular sleeve having a proximal end and a distal end with an flange, said split tubular sleeve reinforced with a wire cage with tabs or rings at the distal end, said split tubular sleeve adapted to grip an outer wall of the esophagus through the diaphragm, said flange attached to the distal end of tubular sleeve with a proximal side adapted to abut the inner wall of the stomach;
resecting a lower phrenoesophageal ligament; and,
positioning the prosthetic gastroesophageal collar assembly about the base of the esophagus with the flange abutting the inner wall of the stomach.
